(19) United States
(12) Patent Application Publication
(10) Pub. No.: US 2022/0399079 A1
(43) Pub. Date: Dec. 15, 2022

(54) METHOD AND SYSTEM FOR COMBINED DNA-RNA SEQUENCING ANALYSIS TO ENHANCE VARIANT-CALLING PERFORMANCE AND CHARACTERIZE VARIANT EXPRESSION STATUS

(71) Applicant: KONINKLIJKE PHILIPS N.V., EINDHOVEN (NL)

(72) Inventors: Yee Him CHEUNG, Boston, MA (US); Jie WU, Cambridge, MA (US); Nevenka DIMITROVA, Pelham Manor, NY (US)

(21) Appl. No.: 17/776,119

(22) PCT Filed: Nov. 5, 2020
(86) PCT No.: PCT/EP2020/081032

§ 371 Date: May 11, 2022

Related U.S. Application Data
(60) Provisional application No. 62/934,120, filed on Nov. 12, 2019.

(51) Int. Cl. G16B 30/10; G16B 20/20

(52) U.S. Cl. CPC G16B 30/10; G16B 20/20

(57) ABSTRACT

A method (100) for characterizing variant expression status for variants identified from a genomic sample, comprising: (i) obtaining (110) DNA sequencing data for the genomic sample; (ii) obtaining (110) RNA sequencing data for the genomic sample, wherein the obtained RNA sequencing data further comprises expression data for each variant; (iii) merging (130) the aligned DNA and RNA sequencing data into a merged alignment; (iv) identifying (140) a plurality of variants relative to the reference genome to generate a set of variants; (v) characterizing (150) an RNA-editing and/or expression status for each of at least a plurality of variants, wherein the expression status comprises one of a plurality of allele-specific expression categorizations comprising expression information for an alternative allele of the variant and expression information for a reference allele of the variant if there is one; and (vi) generating (160) a report comprising the characterized expression status for the variants.

### FIELD OF THE DISCLOSURE

The present disclosure is directed generally to methods and systems for improving genetic variant calls and characterizing variant expression.

### BACKGROUND

As technology for utilizing different types of molecular information becomes more accessible at a lower cost, it is becoming more common to generate multiple types of -omic data (e.g., genomic, transcriptomic, proteomic, and epigenomic) for the same sample. This enables better understanding of the underlying complex biological system. The launch of commercial assays such as the NanoString® Vantage 3D and the Illumina® TruSight Tumor 170, based respectively on nCounter® and next-generation sequencing (NGS) technologies, which support the simultaneous extraction of DNA, RNA, and even protein data, pushes further the demand for multi-omic data analysis. While the different types of -omic data can be analyzed in separate silos by different bioinformatics pipelines, this mainstream approach fails to take advantage of the functional relationships of this data at the molecular level. It also fails to generate new insights into the functional or even pathological impacts of individual aberrations.

DNA and RNA sequencing are the major techniques use to profile the genome and transcriptome. DNA sequencing is mainly used for variant calling, and RNA sequencing is primarily used to measure gene and transcript expression levels. However, mutations such as single nucleotide variants (SNVs) can also be obtained from RNA sequencing data and the information of RNA sequencing variants is similar to that of DNA sequencing variants. For the detection of gene fusions in particular, RNA sequencing is actually the mainstream approach. Use of RNA sequencing data in this manner provides the opportunity to cross-validate or improve the detection of mutations, and furthermore to investigate their transcriptional abundance and functional impacts. This improvement is necessary, as variant calls can be problematic and their functions largely unknown. Indeed, factors such as sample quality, experimental procedures and sequencing coverage, can all influence the variant call quality, sensitivity, and specificity.

### SUMMARY OF THE DISCLOSURE

There is a continued need for methods and systems that integrate multi-omic data to improve variant call and characterization. The present disclosure is directed to inventive methods and systems for characterizing variant expression status for a plurality of variants identified from a genomic sample. Various embodiments and implementations herein are directed to a system and method that merges aligned RNA sequencing data and aligned DNA sequencing data into a single merged alignment. Variants are then identified in the single merged alignment, and a sub-set of variants are identified that satisfy a predetermined minimum read count threshold. The sub-set of variants is characterized using the expression data from the RNA sequencing to assign an RNA-editing and expression status to each variant. The expression status comprises one of a plurality of allele-specific expression categorizations comprising expression information for an alternative allele of the variant and expression information for a reference allele of the variant if there is one. A report is generated that includes the characterized expression status for one or more of the plurality of variants within the sub-set.

Generally, in one aspect, is a method for characterizing variant RNA editing and/or expression status for a plurality of variants identified from a genomic sample, using a variant analysis system. The method includes: (i) obtaining DNA sequencing data for the genomic sample, the DNA sequencing data comprising a plurality of different variant types and aligned to a reference genome to generate aligned DNA sequencing data; (ii) obtaining RNA sequencing data for the genomic sample, the RNA sequencing data comprising a plurality of different variant types and aligned to the reference genome to generate aligned RNA sequencing data, and wherein the obtained RNA sequencing data further comprises expression data for each variant; (iii) merging the aligned RNA sequencing data and aligned DNA sequencing data into a single merged alignment, wherein each read comprises a source identifier; (iv) identifying, in the single merged alignment, a plurality of variants relative to the reference genome, the plurality of variants comprising a plurality of different variant types, to generate a set of variants; (5) characterizing, using the expression data, an RNA editing and/or expression status for each of at least a plurality of variants within the set of variants, wherein the expression status comprises one of a plurality of allele-specific expression categorizations comprising expression information for an alternative allele of the variant and expression information for a reference allele of the variant if there is one; and (vi) generating a report comprising the characterized expression status for the plurality of variants within the set of variants.

According to an embodiment, the plurality of variants is identified using an RNA sequencing data variant calling protocol.

According to an embodiment, the plurality of different variant types comprises at least single nucleotide variants, insertions, deletions, copy number variants, and gene fusions.

According to an embodiment, the obtained RNA sequencing data comprises gene expression data, transcript expression data, exon expression data, splicing data, and/or allele-specific expression data.

According to an embodiment, each of the plurality of allele-specific expression categorizations comprise an identifier describing the expression information for the alternative allele of the variant relative to the expression information for the reference allele of the variant, and wherein there are a plurality of different identifiers. According to an embodiment, the plurality of different identifiers comprise one or more of unexpressed site, unexpressed variant, expressed variant homozygous, expressed variant up regulated, expressed variant down regulated, expressed variant neutral, expressed variant with inconsistency, unexpressed variant with inconsistency, low-confidence RNA-editing, and high-confidence RNA-editing.

According to another aspect is a system for characterizing variant RNA editing and/or expression status for a plurality of variants identified from a genomic sample. The system includes: DNA sequencing data for the genomic sample, the DNA sequencing data comprising a plurality of different variant types and aligned to a reference genome to generate aligned DNA sequencing data; RNA sequencing data for the genomic sample, the RNA sequencing data comprising a plurality of different variant types and aligned to the reference genome to generate aligned RNA sequencing data, and wherein the obtained RNA sequencing data further comprises expression data for each variant; a processor configured to: (i) merge the aligned RNA sequencing data and aligned DNA sequencing data into a single merged alignment; (ii) identify, in the single merged alignment, a plurality of variants relative to the reference genome, the plurality of variants comprising a plurality of different variant types, to generate a set of variants; (iii) characterize, using the expression data, an RNA editing and/or expression status for each of at least a plurality of variants within the set of variants, wherein the expression status comprises one of a plurality of allele-specific expression categorizations comprising expression information for an alternative allele of the variant and expression information for a reference allele of the variant if there is one; and (iv) generate a report comprising the characterized RNA editing and/or expression status for the plurality of variants within the set of variants; and a user interface configured to provide the generated report.

According to another aspect is a method for characterizing variant RNA editing and/or expression status for a plurality of variants identified from a genomic sample, using a variant analysis system. The method includes: (i) obtaining DNA sequencing data for the genomic sample, the DNA sequencing data comprising a plurality of different variant types and aligned to a reference genome to generate aligned DNA sequencing data; (ii) obtaining RNA sequencing data for the genomic sample, the RNA sequencing data comprising a plurality of different variant types and aligned to the reference genome to generate aligned RNA sequencing data, and wherein the obtained RNA sequencing data further comprises expression data for each variant; (iii) identifying, a plurality of variants in the DNA sequencing data and a plurality of variants in the RNA sequencing data, each of the plurality of variants comprising a plurality of different variant types, to generate a set of DNA variants and a set of RNA variants; (iv) merging the set of DNA variants and the set of RNA variants into a single set of variants, or validating the plurality of variants in the DNA sequencing data or the plurality of variants in the RNA sequencing data with the variants in the other sequencing data type, to generate a single set of variants; (v) characterizing, using the expression data, an RNA editing and/or expression status for each of at least a plurality of variants within the set of variants, wherein the expression status comprises one of a plurality of allele-specific expression categorizations comprising expression information for an alternative allele of the variant and expression information for a reference allele of the variant if there is one; and (vi) generating a report comprising the characterized RNA editing and/or expression status for the plurality of variants within the set of variants.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for improving variant detection and characterizing variant expression status of variants in a genomic sample, in accordance with an embodiment.

FIG. 2 is a flowchart of a method for improving variant detection and characterizing variant expression status of variants in a genomic sample, in accordance with an embodiment.

FIG. 3 is a flowchart of a method for improving variant detection and characterizing variant expression status of variants in a genomic sample, in accordance with an embodiment.

FIG. 4 is a flowchart of a method for improving variant detection and characterizing variant expression status of variants in a genomic sample, in accordance with an embodiment.

FIG. 5 is a flowchart of a method for characterizing allele-specific expression of a variant, in accordance with an embodiment.

FIG. 6A is an example of RNA (upper track) and DNA (lower track) read alignment of a false-positive RNA-editing variant due to low DNA coverage, in accordance with an embodiment.

FIG. 6B is an example of RNA (upper track) and DNA (lower track) read alignment of a false-positive RNA-editing variant due to low-quality or ambiguous DNA reads, in accordance with an embodiment.

FIG. 6C is an example of RNA (upper track) and DNA (lower track) read alignment of a true RNA-editing variant supported by a sufficient number of good-quality DNA reads, in accordance with an embodiment.

FIG. 7 is a flowchart of a method for analyzing possible RNA edits, in accordance with an embodiment.

FIG. 8 is a schematic representation of a system for analyzing a genome, in accordance with an embodiment.

### DETAILED DESCRIPTION OF EMBODIMENTS

The present disclosure describes various embodiments of a system and method to assign an RNA editing and expression status to variants identified in merged DNA and RNA sequencing. More generally, Applicant has recognized and appreciated that it would be beneficial to provide a method that integrates DNA and RNA sequencing data to improve variant call and characterization. The system, which may optionally comprise a sequencing platform, generates or receives DNA sequencing data and RNA sequencing data for a genomic sample. The system merges aligned DNA sequencing data and aligned RNA sequencing data into a single merged alignment. Variants are then identified in the single merged alignment, and a sub-set of variants are identified that satisfy a predetermined minimum count threshold of reads with sufficient quality. The system characterizes each variant in the sub-set using the expression data from the RNA sequencing to assign an RNA editing and expression status to each variant. The expression status comprises one of a plurality of allele-specific expression categorizations comprising expression information for an alternative allele of the variant and expression information for a reference allele of the variant if there is one. The system then generates a report that includes the characterized expression status for one or more of the plurality of variants within the sub-set.

According to an embodiment, the approaches described or otherwise envisioned herein may: (i) improve variant calling accuracy by merging variants, including SNVs, indels, fusions detected from multiple data sources, such as DNA-Seq and RNA-Seq data, with quality control (QC) filtering, and (ii) provide information on the RNA-editing and expression status of each variant, such as unexpressed site/variant, expressed variant—homozygous/up-regulated/down-regulated/neutral, expressed/unexpressed variant with inconsistency, and high/low-confidence RNA-editing, based on their allele specific read count or expression, among other improvements and goals.

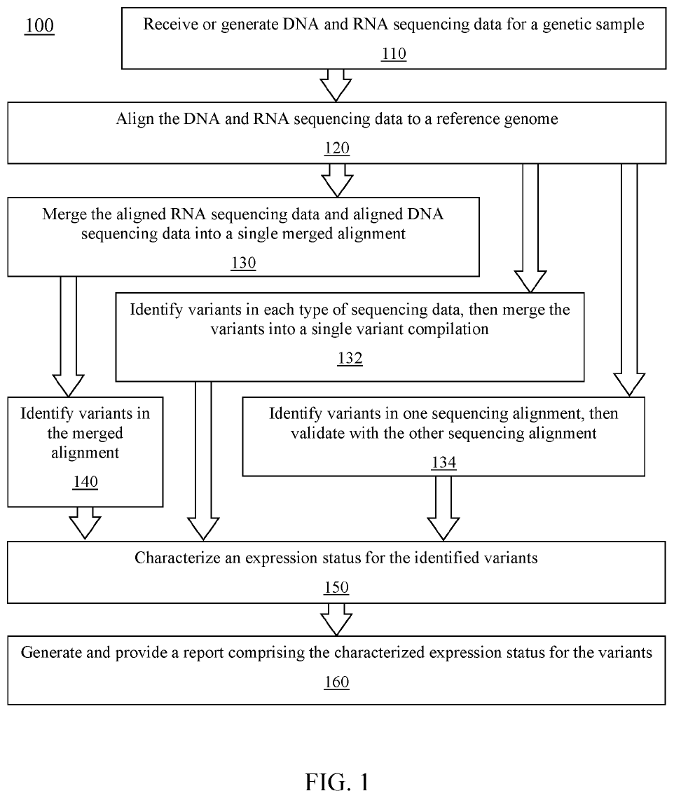

Referring to FIG. 1, in one embodiment, is a flowchart of a method 100 for characterizing variant expression status of variants in a genomic sample using a variant analysis system. The variant analysis system may be any of the systems described or otherwise envisioned herein, and may comprise any of the components described or otherwise envisioned herein.

At step 110 of the method, the variant analysis system generates and/or receives DNA and RNA sequencing data for a genetic sample. The genetic sample can be any genetic sample from any organism, including humans, pathogenic and non-pathogenic organisms, and many. It is recognized that there is no limitation to the source of the genetic sample.

According to an embodiment, the variant analysis system comprises a DNA and/or RNA sequencing platform configured to obtain sequencing data from the genetic sample. The sequencing platform can be any sequencing platform, including but not limited to any system described or otherwise envisioned herein. A sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner. According to an embodiment, the variant analysis system receives the DNA and/or RNA sequencing data for the genetic sample. For example, the variant analysis system may be in communication or otherwise receive DNA and/or RNA sequencing data from a database comprising one or more genetic samples.

The generated and/or received DNA and/or RNA sequencing data may be stored in a local or remote database for use by the variant analysis system. For example, the variant analysis system may comprise a database to store the DNA and/or RNA sequencing data for the genetic sample, and/or may be in communication with a database storing the sequencing data. These databases may be located with or within the variant analysis system or may be located remote from the variant analysis system, such as in cloud storage and/or other remote storage.

The generated and/or received DNA and/or RNA sequencing data may comprise a complete or mostly complete genome, or may be a partial genome, or may be a small portion of a genome. For example, the generated and/or received sequencing data may be assemblies, whole genome constructs, incomplete genomes, partial genomes, exomes, and/or any other sequencing data.

The generated and/or received DNA and/or RNA sequencing data each comprise a plurality of different variant types, including but not limited to single nucleotide variants, insertions, deletions, copy number variants, and gene fusions. Many other variant types are possible. Gene fusions may be detected using a variety of systems, including but not limited to dRanger with Breakpointer, FusionMap, and/or other tools. Other structural variants such inversions, translocations, and others may be detected using a variety of systems, including but not limited to SVDetect, BreakDancer, and/or other tools.

The generated and/or received RNA sequencing data also comprises expression data for each variant, including but not limited to gene expression data, transcript expression data, exon expression data, splicing data, and/or allele-specific expression data. The expression data is obtained, analyzed, reported, and/or stored using any method utilized to do so from RNA sequencing data. The expression data can comprise information about allele-specific expression (ASE); allele-specific splicing (ASS); exon, transcript and gene (including long non-coding RNA, i.e. lncRNA) expressions; differential exon, transcript and gene (including lncRNA) expressions, either based on comparison with a matched normal sample and/or average expressions and their standard deviations in unrelated normal tissues; and/or gene pathway activity prediction by running methods such as Philips OncoSignal and other methods on gene expression and other required data.

If the source is a germline, obtained data may include the genotype (such as homozygous major, heterozygous, homozygous minor), copy number (which could be compared with healthy population of the same background), and/or other information. If the source is somatic, obtained data may include variant allele frequency (VAF), differential copy number variation (compared with matched or unrelated normal tissues), and/or other information.

At optional step 120 of the method, the DNA and RNA sequencing data is aligned to a reference genome with quality checking. The reference genome used for the alignment may be any reference genome, such as a standard reference genome or a reference genome selected from a plurality of possible reference genomes. The reference genome may be obtained from a public or private database or storehouse of reference genomes, and may be in any format utilizable by the variant analysis system. According to an embodiment, the reference genome is a FASTA file, although many other file types are possible. Among other possibilities, the reference genome may be a graph-based genome. The sequencing data, comprising a plurality of sequencing reads, is aligned with the reference genome using any method of alignment, including but not limited to current and future alignment algorithms or methods. There are a variety of different tools available for sequence alignment, including both proprietary and open-source software, and any of these tools may be used to align the plurality of sequencing reads to the reference genome. The DNA sequencing data and the RNA sequencing data may be aligned to a reference genome separately.

This step may be optional, for example, if the DNA and/or RNA sequencing data are obtained from a source or database in which the data has already been aligned to a reference database.

At step 130 of the method, the aligned RNA sequencing data and the aligned DNA sequencing data are merged into a single merged alignment. Thus, there will be a single alignment file with the aligned reads from both the RNA sequencing data and the DNA sequencing data. The RNA sequencing data and DNA sequencing data may be combined into a single alignment file using any method for collating read data from more than one file.

According to an embodiment, each read is associated with an identifier that provides the origination of the read, such that after variant calling each read can be traced back to a source or original file. For example, reads from RNA sequencing data can be associated with an identifier that indicates RNA sequencing as the source of the read. Similarly, reads from DNA sequencing data can be associated with an identifier that indicates DNA sequencing as the source of the read.

At step 140 of the method, variants are identified in the single merged alignment. Thus, variants are identified using both the RNA sequencing and RNA sequencing data in a single alignment. Variants may be identified using any variant calling algorithm, including but not limited to Varscan®, Samtools, and GATK®, among many others. For each variant, the variant calling algorithm may identify, for example, the location of the allele variant, the variant alleles at that location, and/or the frequencies of the variant alleles at that location. The variant alleles will typically comprise one allele corresponding to the reference genome (a “reference allele”) and a second, different allele (a “non-reference allele” or “alternative allele”).

While genomic variants are mainly detected from DNA sequencing data, variants that are expressed (i.e., transcribed into RNA) can also be detected using RNA sequencing data. In general, variant information extracted from RNA sequencing is similar to that of the DNA sequencing. There can be some differences, in some embodiments, when using RNA sequencing data. For example, mutations may be mostly located in expressed regions (i.e., mainly exonic regions), although sometimes mutations may also present in intronic regions (i.e., unspliced transcript), intergenic regions (such as DNA contaminations). These may be a minor portion with good quality data. Additionally, highly expressed genes will have better coverage for mutation calling, while in DNA sequencing data, coverage is relatively more uniform. For the detection of gene fusions in particular, RNA sequencing is actually the mainstream approach using tools such as TopHat-Fusion, STAR-fusion, and others. The ability to detect CNVs from RNA sequencing may be limited to larger-scale CNVs. According to an embodiment, with proper quality control (QC) of the variant calls, the detection accuracy will be improved by detecting variants after merging the DNA sequencing data and the RNA sequencing data into a single merged alignment file.

According to an embodiment, the determination of a variant at a location may be determined in part based on a pre-determined or variable threshold. Thus, a location may be determined to be a variant only if there is a high-confidence variant identified at that location. The variant calling algorithm may, for example, require that a variant be identified in a minimum percentage of high-quality reads aligned at a location, where the minimum percentage can be or can be based on a pre-determined or variable threshold. The threshold may be programmed into the variant analysis system or may be determined or modified by a user of the variant analysis system, or by another system working with the variant analysis system.

According to an embodiment, for some applications the variant analysis system and/or variant calling algorithm may be programmed or otherwise instructed or designed to require that a variant be identified in at least 25% of reads at a location, such that variants identified in less than 25% of reads are considered noise and won't be identified as variant alleles or a heterozygous location. According to another application, such as one requiring a more stringent variant calling protocol, the variant analysis system and/or variant calling algorithm may be programmed or otherwise instructed or designed to require that a variant be identified in at least 40% of reads at a location. According to an embodiment, the threshold may optionally be wholly or partially dependent upon the read depth at the analyzed location. These and many other thresholds and variations may be programmed, selected, or otherwise determined by the system and/or by a user.

According to an embodiment, the variant analysis system generates an output from the analysis by the variant calling algorithm or method. The output may be, for example, any of the information generated by the variant calling algorithm or method. For example, the output may comprise one or more variant locations and the values of the variant alleles at each location. The output may comprise additional information, including but not limited to the frequencies of the variant alleles at each location, among other types of information. This output may be utilized in downstream functionality of the variant analysis system as described or otherwise envisioned herein.

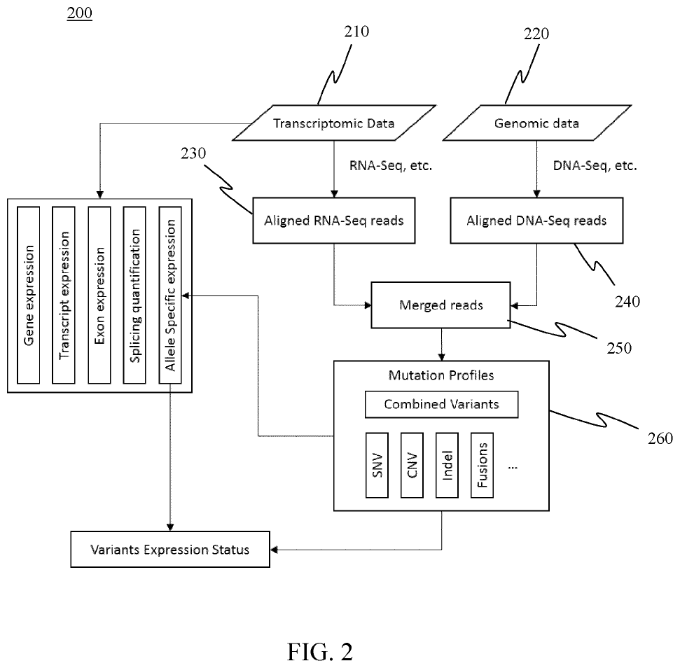

Referring to FIG. 2, in one embodiment, is a flowchart showing a method 200 for characterizing variant expression status of variants in a genomic sample using a variant analysis system. The variant analysis system may be any of the systems described or otherwise envisioned herein, and may comprise any of the components described or otherwise envisioned herein. At 210, RNA sequencing data is obtained from transcriptomic data, and DNA sequencing data is obtained from genomic data at 220. The RNA sequencing data and DNA sequencing data are separately aligned at 230 and 240, as described herein. At 250 the separate alignments are merged to create a single merged alignment. At 260, the single merged alignment is utilized to identify variants in the data.

According to another embodiment, the method progresses from step 120 to step 132 of the method, without performing step 130. In this embodiment, variants are identified separately in the RNA and DNA sequencing alignments. At step 132 of the method, the variant are identified separately in the RNA and DNA sequencing alignments, and then the variants are merged into a single variant file or compilation. Before merging, the RNA and DNA sequencing alignments may include, for example, quality scores and/or statistics such as read count for reference and alternative allele, for each variant. The output of the merge may include, for example, an output file of merged variant calls, quality control statistics for the merged variants, and/or a file or list of discarded variants.

According to an embodiment, quality control analysis may be performed on the lists of variants from different data sources based on their call statistics, quality scores, and user-defined quality criteria. The variants may be identified with a quality status such as ‘Low-quality,’ ‘Med-quality,’ and ‘High-quality,’ among others. A union merge of the variant calls from different data sources may be performed, with an indication in a separate column its source, such as ‘RNA-Seq,’‘DNA-Seq,’ or ‘both.’ Quality information may be added or used from different data sources. A final quality status may be determined on the quality statuses from both sources, or from an individual source. The merged variant calls may be saved, for example, as a “merged_variants” file in a desired output file format. There may also be an output of the quality control data for the merged variants.

According to an embodiment, the merge of variants may involve several considerations. For example, if a variant is supported by multiple sources, its quality can be lifted up to the next level. Similarly, there may be less-stringent filters that can be used to report more variants from each source, since the later combining step will revisit the qualities, and more variants can be recovered in this way.

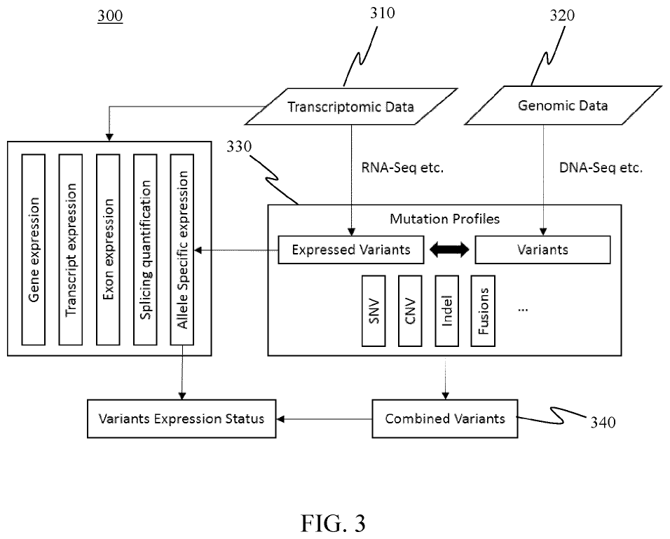

Referring to FIG. 3, in one embodiment, is a flowchart showing a method 300 for characterizing variant expression status of variants in a genomic sample using a variant analysis system. The variant analysis system may be any of the systems described or otherwise envisioned herein, and may comprise any of the components described or otherwise envisioned herein. At 310, RNA sequencing data is obtained from transcriptomic data, and DNA sequencing data is obtained from genomic data at 320. The RNA sequencing data and DNA sequencing data are separately aligned, as described herein, and then variants are separately identified in the separate alignments at 330. At 340, the separate variants are merged into a single variant file, with one or more of the possible outputs described or otherwise envisioned herein.

According to another embodiment, the method progresses from step 120 to step 134 of the method, without performing step 130. In this embodiment, the sequencing data of one type is utilized to validate variant calls from the other type of sequencing data. At step 134 of the method, variants are identified in the DNA or RNA sequencing alignment, and then the other sequencing data (RNA if DNA was first used, or DNA if RNA was first used) is utilized to validate variant calls.

According to an embodiment, input to step 134 may include variant calls from either the DNA-Seq or RNA-Seq data with quality scores and/or statistics such as read count for reference and alternative allele for each variant. The input may also include raw reads from the other technique (i.e., RNA if DNA was first used, or DNA if RNA was first used). The output may be a file of the combined variant calls along with quality control data for the combined variants.

According to an embodiment, instead of calling variants from the raw reads (i.e., RNA sequencing raw reads if DNA was first used, or DNA sequencing raw reads if RNA was first used), the system looks for the variant called from the first source in the second source. If the variant called from the first source is found in the second source, the variant is reported. According to an embodiment, if the variant is found in both sources—that is, if the variant is validated by being found in the second source, it can be labeled quality status such as ‘Low-quality,’ ‘Med-quality,’ and ‘High-quality,’ among others, based in whole or in part on its call statistics, quality scores, and/or user-defined quality criteria.

According to an embodiment, this approach is faster than current approaches and is especially sensitive to very low-quality variants from the second, verifying source. Additionally, this approach may be particularly useful when one knows that the variants called from the first source can be treated as standard. For example, if the first source is high coverage DNA-seq data and the second source is RNA-Seq data from the same sample, in this case, the mutations called from the second source would be an approximate subset (considering RNA editing, etc.). Instead of calling the mutation again, this approach focuses more on validating the known mutations.

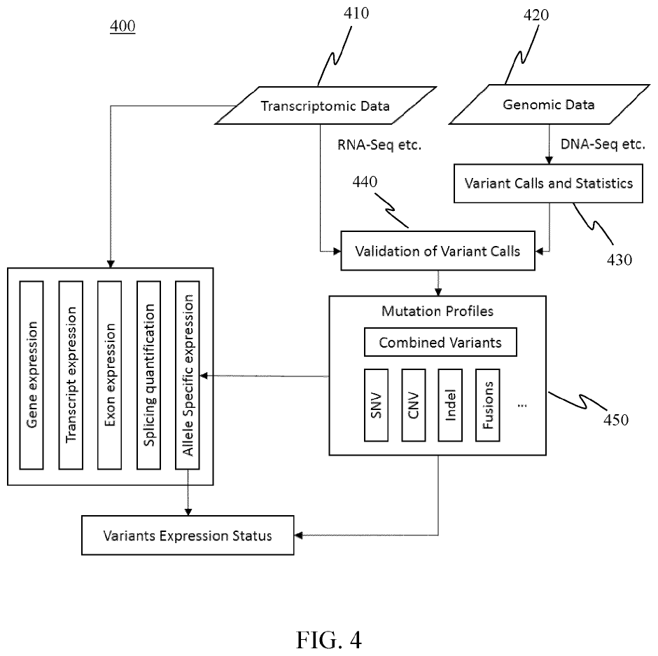

Referring to FIG. 4, in one embodiment, is a flowchart showing a method 400 for characterizing variant expression status of variants in a genomic sample using a variant analysis system. The variant analysis system may be any of the systems described or otherwise envisioned herein, and may comprise any of the components described or otherwise envisioned herein. At 410, RNA sequencing data is obtained from transcriptomic data, and DNA sequencing data is obtained from genomic data at 420. The RNA sequencing data and DNA sequencing data are separately aligned, as described herein, and then variants are identified in the DNA sequencing data at 430. Although FIG. 4 shows variants being identified using the DNA sequencing data at 430 and then being validated using RNA sequencing data at 440, it is appreciated that the reverse may be true. Accordingly, variants may be identified using the RNA sequencing data at 430 and then can be validated using DNA sequencing data at 440. The list of variants that are validated are output as a single file at 450.

Returning to the method described in FIG. 1, at step 150 of the method an expression status is generated or characterized for each of at least a plurality of variants within the set of variants, utilizing expression data. The generated or characterized expression status comprises one of a plurality of allele-specific expression categorizations comprising expression information for an alternative allele of the variant and expression information for a reference allele of the variant, if there is one.

### Expression Status Determination—First Embodiment

According to one embodiment, an expression status is determined for one or more variants within the set of variants using counts of good-quality RNA-Seq reads for reference and alternative alleles, as well as allele specific expression. The expression status comprises a determination of one of a plurality of allele-specific expression categorizations comprising expression information for an alternative allele of the variant and/or expression information for a reference allele of the variant.

According to an embodiment, the counts of good-quality RNA-Seq reads for reference and alternative alleles can be obtained by tools such as ASEReadCounter among other tools, such as with easily customizable read processing options. Quality control processing steps may include removal of reads with low base quality, removal of duplicates, and other analysis or modification of read data.

According to an embodiment, output from step 150 comprises allele-specific expression categorization (“ase_status”) for each variant, with a categorical variable or identifier that indicates the expression status of the variant. The allele-specific expression categorization may comprise a wide variety of different variables, labels, or identifiers. Although certain possible labels are identified herein, it is understood that these are only examples and that the allele-specific expression categorizations are not limited to these examples.

According to an embodiment, the allele-specific expression categorizations may include: (1) “US” which indicates an unexpressed site with neither expression of the reference allele nor the alternative allele; (2) “UV” which indicates an unexpressed variant and expression of the reference allele only; (3) “EV-Hom” which indicates an expressed variant which is homozygous with expression of the alternative allele only; (4) “EV-Up” which indicates an expressed variant with significantly higher expression of the alternative allele relative to the reference allele; (5) “EV-Dn” which indicates an expressed variant with significantly lower expression of the alternative allele relative to the reference allele; and (6) “EV-N” which indicates an expressed variant with similar expression of the alternative allele and the reference allele. According to an embodiment, output from step 150 further comprises the expression or read count of the reference allele (“ase_ref”) and/or the expression or read count of the alternative allele (“ase_alt”), among other possible information.

The allele-specific expression categorizations may be generated using any of a wide variety of possible methods or algorithms. According to an embodiment, the allele-specific expression categorizations are generated using the algorithm described below, although many other methods or algorithms may be utilized. The following is provided only as one example algorithm, and is not intended to limit the possible methods or algorithms that may be utilized.

Pursuant to this allele-specific expression categorization algorithm, the following variables and parameters may be utilized:
nᵣ and nₐ are the number of mapped reads with respectively the reference and alternative alleles;
n is the total number of reads mapped to the variant site, i.e. nᵣ+nₐ;
eᵣ and eₐ are the expression levels of respectively the reference and alternative alleles;
e is the overall expression level of the variant site;
tᵣ, tₐ, and t are the user-defined threshold number of mapped reads for the non-trivial expressions of respectively the reference allele, alternative allele, and variant site in general;
sᵣ, sₐ, and s are the user-defined minimum non-trivial expression levels of respectively the reference allele, alternative allele, and the variant site in general;
v is the VAF of the alternative allele;
vᵤ and u_{d} are the user-defined VAF bounds for respectively the up- or down-regulated expression of the alternative allele;
u is the differential expression level of the alternative allele; and
uᵤ and u_{d} are the user-defined differential expression bounds for respectively the up- or down-differential expression of the alternative allele.

With these variables and parameters, this allele-specific expression categorization algorithm categorizes the expression status via the following analysis:
If n<t or e<s, then ase_status=“US” (i.e. unexpressed site);
Else If nₐ<tₐ or eₐ<sₐ, then ase_status=“UV” (i.e. unexpressed variant);
Else if nᵣ<tᵣ or eᵣ<sᵣ, then ase_status=“EV-Hom” (i.e. expressed variant—homozygous);
Else if v>vᵤ or u>uᵤ, then ase_status=“EV-Up” (i.e. expressed variant—up regulated);
Else if v<v_{d} or u<u_{d}, then ase_status=“EV-Dn” (i.e. expressed variant—down regulated); and
Else ase_status=“EV-N” (i.e. expressed variant—neutral).

Additionally:
ase_ref=nᵣ (or ase_ref=eᵣ); and
ase_alt=nₐ (or ase_alt=eₐ).

Thus, according to an embodiment the output of step 150 is an expression categorization (“ase_status”), as well as the expression or read count of the reference allele (“ase_ref”) and the expression or read count of the alternative allele (“ase_alt”), among other possible information. These and other outputs are possible. One of more of the variables or parameters set forth in the algorithm above may be modified or eliminated to adjust the algorithm, in addition to other possible modifications.

### Expression Status Determination—Second Embodiment

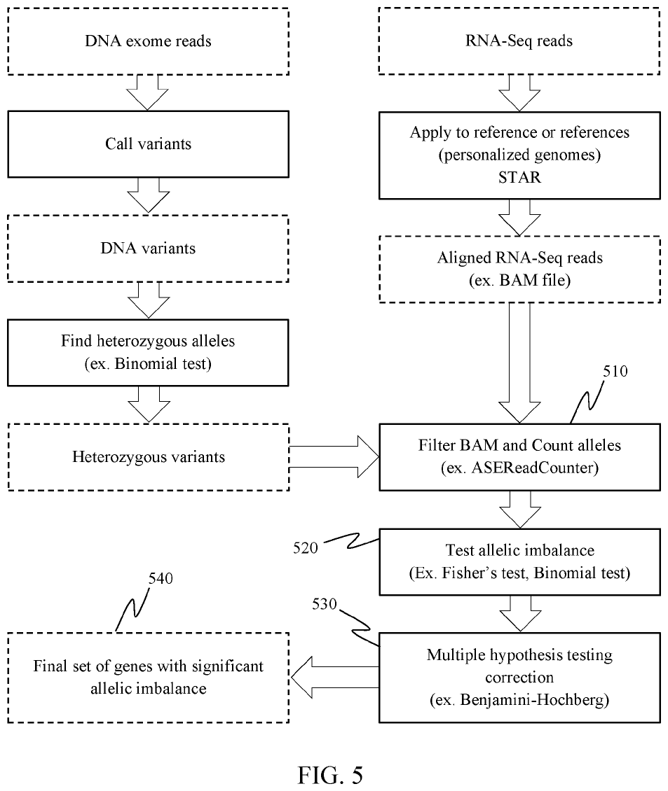

According to another embodiment, the characterized expression status for one or more variants comprises one or more of allele specific expression status, expression status, and/or RNA editing status. According to another embodiment, the characterized expression status comprises an allele specific expression status, an expression status, and an RNA editing status for one or more variants. Referring to FIG. 5 is a flowchart depicting one embodiment of a possible method or algorithm for the detection of allele-specific expression (ASE).

As described or otherwise envisioned herein, DNA-seq and RNA-seq are performed and the analysis proceeds along one or more possible embodiments until a list of variants using the DNA-seq and/or RNA-seq data is generated.

The method utilizes certain inputs for determination of allele specific expression status, expression status, and/or RNA editing status for a variant. The method or system requires a list of the variant calls identified in a previous step of the method. Also required for each of these variants in the list, or at a minimum the variants to be analyzed from the list which may be some or all of the variants, is certain information used for the determination. This can include, for example, one or more of: (1) a count of RNA-Seq reads for the reference and variant allele at step 510; (2) a count of DNA-Seq reads for the reference and variant allele at step 510; (3) the overall expression level for the reference and variant allele, optionally including information about the allele-specific expression such as the name of the method and the associated parameters for the evaluation of allele-specific expression (such as Binomial or Fisher), and/or a list of the parameters involved in the classification of expression status, at step 520; and/or (4) multiple hypothesis testing correction at step 530; to create a final set of genes or variants at 540. According to an embodiment there is a pointer or other link to or information about the DNA-Seq read alignment file for the variant and/or reference allele used for confirmation of RNA-editing variants.

Count of RNA-Seq Reads

According to an embodiment, the count of RNA-Seq reads for the reference and variant allele may be a count of all reads or a count of reads that satisfy a predetermined quality threshold. Thus, the count of RNA-Seq reads may comprise good-quality RNA-Seq reads for the reference allele (rna_ref) and variant allele (rna_alt) for the variant or for any other location in the genome. The RNA-Seq read count for reference and alternative alleles can be obtained by tools such as ASEReadCounter among other tools, such as with easily customizable read processing options, analyzing aligned reads in BAM files or other types of alignment files. Quality control processing steps may include removal of reads with low base quality, removal of duplicates, and other analysis or modification of read data. According to an embodiment, “good-quality RNA-seq reads” may be defined as reads that pass the quality-check of the read alignment tool or those with alignment scores higher than a predetermined threshold. The predetermined threshold may be determined or programmed by the software, hardware, and/or a user, among other possibilities.

Count of DNA-Seq Reads

According to an embodiment, the count of DNA-Seq reads for the reference and variant allele may be a count of all reads or a count of reads that satisfy a predetermined quality threshold. Thus, the count of DNA-Seq reads may comprise good-quality DNA-Seq reads for the reference allele (dna_ref) and variant allele (dna_alt) for the variant or for any other location in the genome. The DNA-Seq read count for reference and alternative alleles can be obtained by tools such as Samtools/BCFtools among other tools, analyzing aligned reads in BAM files or other types of alignment files. According to an embodiment, “good-quality DNA-seq reads” may be defined as reads that pass the quality-check of the read alignment tool or those with alignment scores higher than a predetermined threshold. The predetermined threshold may be determined or programmed by the software, hardware, and/or a user, among other possibilities.

According to an embodiment, the method utilizes overall expression level (expr) of the reference and/or alternative alleles for the variant. The system may also use information about the name of the method, and associated parameters, utilized to evaluate or determine allele specific expression (ASE). For example, methods such as ‘Binomial’ or ‘Fisher’ may be utilized to determine or evaluate ASE. The system may also use or comprise information about the parameters involved in the classification of expression status for the variant. The system may further comprise or use a pointer or other link to or information about the DNA-Seq read alignment file for the variant and/or reference allele used for confirmation of RNA-editing variants.

The step of generating or characterizing an expression status for one or more variants comprises, for example, detection of allele specific expression. There are numerous approaches for detecting allele specific expression, and the method or system may utilize any of those approaches. Although several approaches are described herein, it is understood that these approaches are only provided as non-limiting examples only.

According to one approach, the ASE is detected with respect to a predefined ratio using a binomial test. The predefined ratio of reference reads and alternative alleles may be, for example 1:1 or other possible ratio. For example, one method to detect allelic imbalance is to apply Binomial test to evaluate if the ratio:

$$r = \frac{rna\_alt}{rna\_ref + rna\_alt} \qquad ( Eq . 1 )$$

has significantly deviated from an expected value, most often predefined as 0.5, assuming the null hypothesis of an equal number of reads carrying the reference and alternative alleles.

According to another approach, the ASE is detected relative to observed DNA counts using Fisher's exact test. If the goal is to detect ASE attributed exclusively to the transcription process, then the effects of the following factors have to be removed in the analysis: (1) mapping bias, where the reference allele has a higher probability of mapping to the correct position on the reference genome than the alternative allele; and/or (2) copy number variations (CNV) at the DNA level that causes an imbalance in RNA transcripts. The real cause of variation here is a copy number variation and it is undesirable to identify this as ASE. According to an embodiment, the ASE can be detected by applying Fisher's Exact Test to evaluate whether the ratio (rna_alt:rna_ref) of the RNA reads has significantly deviated from the ratio (dna_alt:dna_ref) of the DNA reads. The reasoning behind this is that mapping bias and copy number variations affect both the DNA and RNA counts, so by comparing the RNA counts against the DNA counts these confounding effects can be removed. According to an embodiment, the 2×2 contingency table as input to the Fisher's Exact Test in this case should consist of read counts for the reference and alternative alleles in the columns, and read counts for DNA and RNA data in the rows.

According to an embodiment, after computing the p values of all heterozygous DNA variants, those with significant statistical evidence for ASE can then be identified with multiple hypothesis testing correction such as Bonferroni or false discovery rate (FDR) adjustment, among other options.

ASE Classification/Categorization

According to an embodiment, the method determines the expression status of each variant based on the results of the ASE test just above. The expression status can comprise, for example, a classification or categorization. The expression status classification or categorization may be generated using any of a wide variety of possible methods or algorithms. According to an embodiment, the expression status classification or categorization is generated using the algorithm described below, although many other methods or algorithms may be utilized. The following is provided only as one example algorithm, and is not intended to limit the possible methods or algorithms that may be utilized.

Pursuant to this expression status classification or categorization algorithm, the following variables and parameters may be utilized:
mᵣ, mₐ and m—numbers of mapped DNA reads for respectively the reference allele, alternative allele and variant site in general, where m=mᵣ+mₐ;
nᵣ, nₐ and n—numbers of mapped RNA reads for respectively the reference allele, alternative allele and overall variant site, where n=nᵣ+nₐ;
nᵣ, nₐ′ and n′—threshold numbers of mapped RNA reads for the non-trivial expressions of respectively the reference allele, alternative allele and overall variant site;
eᵣ, eₐ and e—expression levels for respectively the reference allele, alternative allele and overall variant site;
e′—minimum non-trivial expression level of the variant site;
e_{a-H}, e_{a-L}—bounds for respectively the high and low levels of expression for the alternative allele;
p—the adjusted p value for ASE;
p′—the p value threshold for statistical significance of ASE;
s—score that measures the degree of ASE; and
sᵤ, sₙ and s_{d}—bounds for respectively the up/neutral/down regulation of the alternative allele.

According to an embodiment, if ASE is detected based on the Binomial method, then the ASE score can simply be measured by the fraction of reads for the alternative allele, i.e., s=nₐ/n, which ranges from 0 which suggests decreased ASE with no read for the alternative allele to 1 which suggests increased ASE with all reads for the alternative allele.

According to an embodiment, if ASE is detected based on the Fisher's Exact Test with regard to the DNA data, then the ASE score can be defined as

$$s = \frac{{n}_{a}}{n} - \frac{{m}_{a}}{m} ,$$

which ranges from −1 which suggests extreme down-regulation to 1 which suggests extreme up-regulation.

According to an embodiment, the expression level of the alternative allele can be defined as

$${e}_{a} = \frac{{n}_{a}}{n} \cdot e ,$$

although other definitions are possible.

According to an embodiment, the ASE status classification rules may comprise the following, although many other rules are possible:
If p<p′ then there is a statistically significant allelic imbalance;
If s>sᵤ, then ase_status=“Up-S” (strongly up-regulated alternative allele);
   Else if s<s_{d}, then ase_status=“Dn-S” (strongly down-regulated alternative allele);
   Else if s>sₙ, then ase_status=“Up” (moderately up-regulated alternative allele);
   Else ase_status=“Dn” (moderately down-regulated alternative allele);
Else ase_status=“N” (no significant allelic imbalance).

According to an embodiment, the expression status classification may comprise the following, although many other rules are possible:
If n<n′ or e<e′, then expr_status=“U” (uexpressed variant site);
Else if eₐ>e_{a-H}, then expr_status=“H” (high-level expression of the alternative allele);
Else if eₐ<e_{a-L}, then expr_status=“L” (low-level expression of the alternative allele);
Else expr_status=“M” (medium-level expression of the alternative allele).

Thus, according to an embodiment the output at step 540 is a list of variants at least some of which are associated with: (1) an indication of the allelic imbalance ranging from no significant allelic imbalance to different statistically significant allelic imbalances (strongly up-regulated, strongly down-regulated, moderately up-regulated, moderately down-regulated, and so on); and (2) an expression status classification ranging from unexpressed variant site to different expression categories (high-level expression, medium-level expression, low-level expression, and so on). These and other outputs are possible. One of more of the variables or parameters set forth in the algorithm above may be modified or eliminated to adjust the algorithm, in addition to other possible modifications. According to an embodiment, both (1) ASE and (2) expression are measured quantitatively. For ASE, the quantitative measurement is the score just above. For expression, the quantitative measurement is the number of reads nₐ or expression level eₐ of the alternative allele.

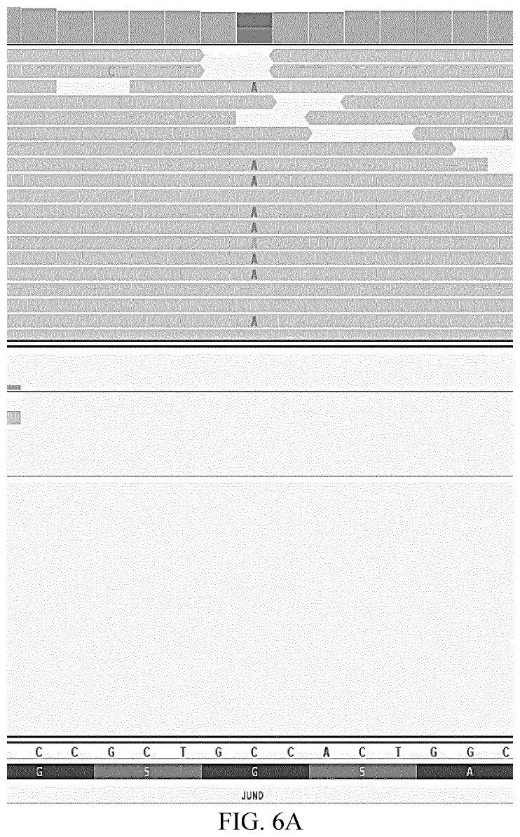

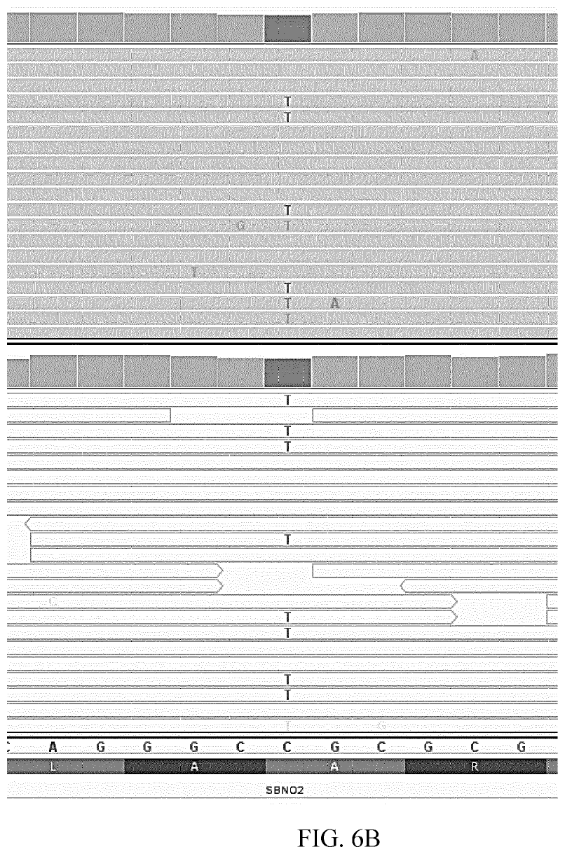

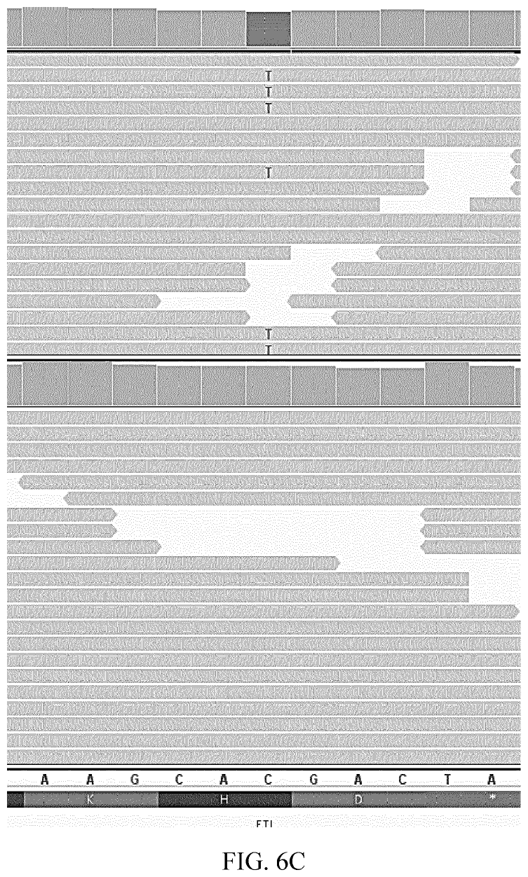

According to an embodiment, the characterized expression status comprises an RNA editing status for one or more variants. RNA editing represents after-transcription modifications to RNA, in other words, mutations that are present in RNA but not in DNA. For this reason, RNA-editing can only be discovered through the integrative analysis of DNA and RNA data. The first step in RNA-editing discovery is to identify variants found only in RNA. While these variants are potential candidates, not being able to find a corresponding mutation in DNA could also be due to low coverage (as shown in FIG. 6A) or low-quality/ambiguous DNA reads (as shown in FIG. 6B). Indeed, referring to FIG. 6 are several different examples of RNA-only variants, with the upper track showing the aligned RNA reads, lower track showing the DNA reads. Both FIG. 6A and FIG. 6B are false-positive examples, due respectively to low DNA coverage and ambiguous reads. Only FIG. 6C shows a true RNA-editing variant.

According to an embodiment, some additional steps can be applied on the candidate variants in order to reduce the number of false positives. For example, the method or system can double-check the corresponding DNA region for any sign of variants by relaxing the criteria for DNA read filtering. For example in FIG. 6B, although the variant exists in DNA, it is only found in ambiguous (transparent) reads and is hence not properly called. If the variant is found in DNA after relaxing the filtering criteria, then there is a report of no RNA editing (rna_edit_status=“N”). As another example, the method or system can check to determine whether the site of the variant has sufficient read coverage of the reference allele in the DNA-seq data. Thus, if the coverage is insufficient, then there is a report of no RNA editing (rna_edit_status=“N”). If the coverage is sufficient but low then there is a report of a low-confidence RNA-edit (rna_edit_status=“LC”). If the coverage is high, then there is a report of a high-confidence RNA-edit (rna_edit_status=“HC”). Many other classifications are possible.

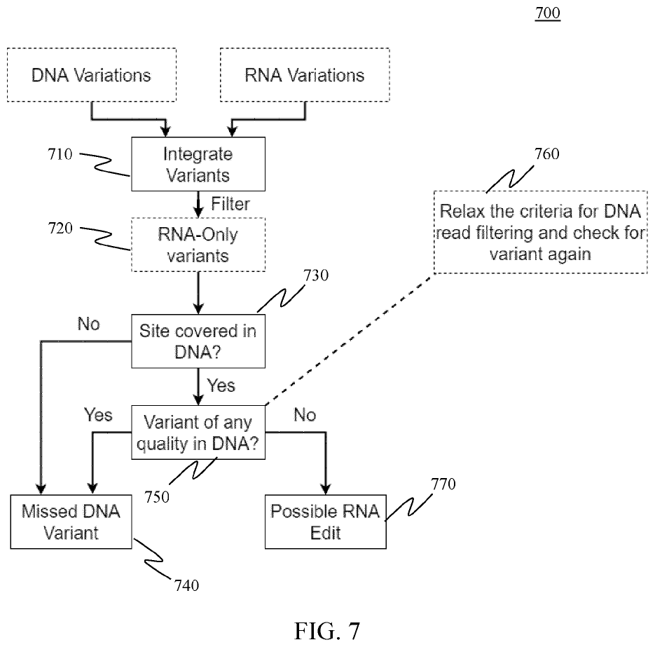

Referring to FIG. 7 is a flowchart of a procedure or method 700 for the identification of RNA-editing variants. At 710, DNA variants and RNA variants are integrated using any of the methods or embodiments described or otherwise envisioned herein. At 720, RNA-only variants are identified. At 730, for one or more of the identified RNA-only variants the system or method determines whether the site of the RNA-only variant is covered by any of the DNA-seq data. At 740, if the site is not covered by any of the DNA-seq data, then there is a report of no RNA editing (rna_edit_status=“N”) and/or a report of a missed DNA variant. If the site is covered by the DNA-seq data, the system or method determines whether the site of the variant has sufficient read coverage of the reference allele in the DNA-seq data at 750, and/or whether the variant is present in the DNA-seq data. If the read coverage is insufficient or the read coverage is sufficient and the variant is present in the DNA-seq data, then at 740 there is a report of a missed DNA variant. If the read coverage is sufficient and the variant is not present in the DNA-seq data at, then at 770 there is a report of a possible RNA edit. As an optional intermediate step 760, the system or method can relax the criteria for DNA read filtering, and then can return to step 750 to determine—using the relaxed criteria and revised DNA-seq data—whether the site of the variant has sufficient read coverage of the reference allele in the DNA-seq data at 750, and/or whether the variant is present in the DNA-seq data.

According to this second embodiment of step 150 of the method 100 in FIG. 1, the output of step 150 is an expression status for the identified variants. This characterized expression status may comprise an allele specific expression status, an expression status, and/or an RNA editing status for one or more variants. The allele specific expression status, expression status, and/or RNA editing status may each optionally comprise additional information.

For example, according to an embodiment the allele specific expression status may comprise a categorization of the regulation of the allele. Thus, the specific expression status may comprise one or more of the following:
ase_status—a categorical variable that indicates the ASE status of a heterozygous DNA variant as one of the following categories among other possible categories:
   N=no ASE or neutral regulation;
   Up=confirmed ASE, with up-regulated alternative allele;
   Up-S=confirmed ASE, with strongly up-regulated alternative allele;
   Dn=confirmed ASE, with down-regulated alternative allele; and/or
   Dn-S=confirmed ASE, with strongly down-regulated alternative allele.
ase_score, ase_pval—score that measures the degree of ASE and two-sided p value for its statistical significance.

Similarly for example, according to an embodiment the expression status may comprise a categorization of the expression status of a variant. Thus, the expression status may comprise one or more of the following among other possible categories:
expr_status—a categorical variable that indicates the expression status of a variant as one of the following categories among other possible categories:
   H=high-level expression of the alternative allele;
   M=medium-level expression of the alternative allele;
   L=low-level expression of the alternative allele; and/or
   U=unexpressed variant site.
expr_alt—expression level of the alternative allele.

Similarly for example, according to an embodiment the RNA editing status may comprise an identification of a variant in RNA data. Thus, the RNA editing status may comprise one or more of the following among other possible categories:
rna_edit_status—a categorical variable that indicate if new variants are detected in RNA data, which may be the following among other possible categories:
   N=no RNA-editing;
   LC=low-Confidence RNA-Edit (Low Coverage in DNA-Seq reads); and/or
   HC=high-Confidence RNA-Edit (High Coverage in DNA-Seq reads).
rna_edit_allele—an identification of the RNA-editing allele if it exists.

These and many other categories and labels are possible for the output of step 150 of the method.

At step 160 of the method, a report comprising the characterized expression status for one, some, or all of the plurality of variants within the set of variants is generated and reported. The report may comprise, for example, the expression categorization, the expression or read count of the reference allele, and/or the expression or read count of the alternative allele, among other possible information. The report may be electronic or printed, and may be stored. For example, the report may comprise a text-based file or other format. The report may comprise a database which is searchable for a particular variant or genomic location. The report may be sortable or otherwise configured for organization to allow easy analysis and extraction of information.

According to an embodiment, the variant analysis system may visually display information about one or more of the variants and characterized expression status on a screen or other display method. A clinician or researcher may only be interested in one or several variants, and thus the variant analysis system may be instructed or otherwise designed or programmed to only display information obtained for the one or several variants.

According to an embodiment, the report or information may be stored in temporary and/or long-term memory or other storage. Additionally and/or alternatively, the report or information may be communicated or otherwise transmitted to another system, recipient, process, device, and/or other local or remote location.

According to an embodiment, once the report or information is generated, it can be provided to a researcher, clinician, or other user to review and implement an action or response based on the provided information. For example, a researcher or clinician may utilize the information to mine for variants in a genetic sample, such as a genome of a patient or a research subject. The user may manually review the report to review all variants, or to identify specific variants through filtering and ranking based on their ASE/expression/RNA-editing status and scores, or may use software or other methodology to identify one or more variants. Identifying variants is an important aspect of disease research, disease diagnosis, and disease treatment. Accordingly a clinician may, for example, diagnose a genetic disorder or hypothesize the existence of a particular genetic disorder based on the output of the report. The clinician may additional or alternatively select a specific treatment based on the output of the report.

As another example, a user may review the report or information to determine whether specific locations within the target genome comprise a variant. For example, a researcher, clinician, or other user may be interested in specific variant alleles for research, treatment, or other purposes and may review a report and/or generate a report directed to the allele locations of interest. The existence or absence of a variant, as indicated by the report, provides the necessary research or treatment information for the user. Many other downstream uses are possible.

Variants are typically called using DNA-seq data sequenced from whole genome or whole exome, for example, somatic mutations are called by comparing patient tumor versus matched normal tissues. However, biological and technical noise makes it difficult to obtain sensitive and accurate results. Pursuant to the methods and systems described or otherwise envisioned herein, sequencing data from multiple sources is used to enhance variant calling accuracy and sensitivity, which significantly improves clinical usability. Indeed, using multiple sources to call mutations adds an additional layer of validation of the variants. DNA and RNA samples are prepared in different ways, and lower coverage of certain regions of genomes can create difficulties in identifying mutations, especially those with lower allele frequencies. However, these regions can be amplified at higher depths in RNA-Seq data, which provides more evidences of the mutations. In other words, combining reads from RNA-Seq and DNA-Seq increases the read coverage for variant calling. An immediate next step of combinational mutation calling from both DNA and RNA is to inform the expression status of the mutations, and the function of a mutation can be interpreted after its expression status is identified.

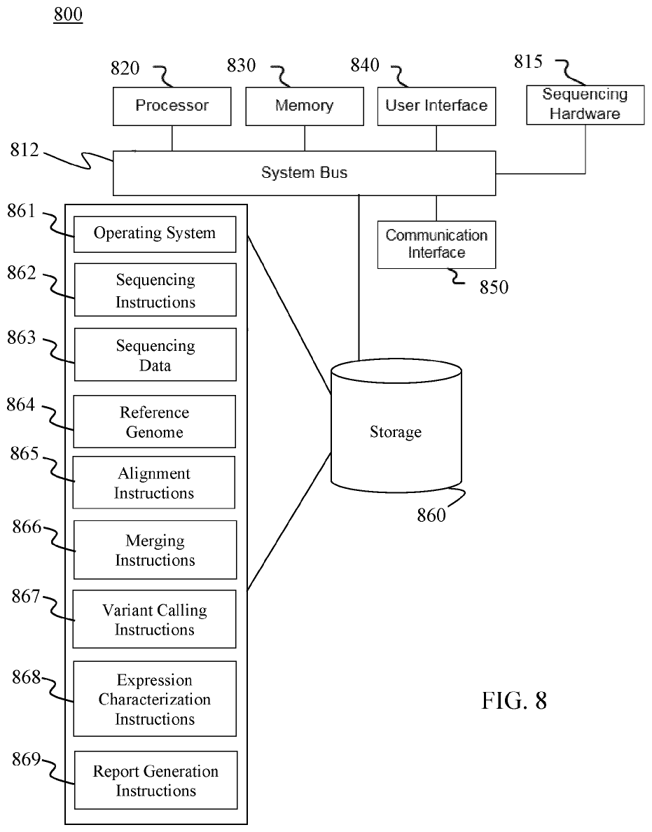

Referring to FIG. 8, in one embodiment, is a schematic representation of a variant analysis system 500 configured to characterize variant expression status for a plurality of variants identified from a genomic sample. System 800 may be any of the systems described or otherwise envisioned herein, and may comprise any of the components described or otherwise envisioned herein.

According to an embodiment, system 800 comprises one or more of a processor 820, memory 830, user interface 840, communications interface 850, and storage 860, interconnected via one or more system buses 812. In some embodiments, such as those where the system comprises or directly implements a DNA and/or RNA sequencer or sequencing platform, the hardware may include additional sequencing hardware 815. It will be understood that FIG. 8 constitutes, in some respects, an abstraction and that the actual organization of the components of the system 500 may be different and more complex than illustrated.

According to an embodiment, system 800 comprises a processor 820 capable of executing instructions stored in memory 830 or storage 860 or otherwise processing data to, for example, perform one or more steps of the method. Processor 820 may be formed of one or multiple modules. Processor 820 may take any suitable form, including but not limited to a microprocessor, microcontroller, multiple microcontrollers, circuitry, field programmable gate array (FPGA), application-specific integrated circuit (ASIC), a single processor, or plural processors.

Memory 830 can take any suitable form, including a non-volatile memory and/or RAM. The memory 830 may include various memories such as, for example L1, L2, or L3 cache or system memory. As such, the memory 830 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 800. It will be apparent that, in embodiments where the processor implements one or more of the functions described herein in hardware, the software described as corresponding to such functionality in other embodiments may be omitted.

User interface 840 may include one or more devices for enabling communication with a user. The user interface can be any device or system that allows information to be conveyed and/or received, and may include a display, a mouse, and/or a keyboard for receiving user commands. In some embodiments, user interface 840 may include a command line interface or graphical user interface that may be presented to a remote terminal via communication interface 850. The user interface may be located with one or more other components of the system, or may located remote from the system and in communication via a wired and/or wireless communications network.

Communication interface 850 may include one or more devices for enabling communication with other hardware devices. For example, communication interface 850 may include a network interface card (NIC) configured to communicate according to the Ethernet protocol. Additionally, communication interface 850 may implement a TCP/IP stack for communication according to the TCP/IP protocols. Various alternative or additional hardware or configurations for communication interface 850 will be apparent.

Storage 860 may include one or more machine-readable storage media such as read-only memory (ROM), random-access memory (RAM), magnetic disk storage media, optical storage media, flash-memory devices, or similar storage media. In various embodiments, storage 860 may store instructions for execution by processor 820 or data upon which processor 820 may operate. For example, storage 860 may store an operating system 861 for controlling various operations of system 800. Where system 800 implements a sequencer and includes sequencing hardware 815, storage 860 may include sequencing instructions 862 for operating the sequencing hardware 815, and sequencing data 863 obtained by the sequencing hardware 815, although sequencing data 863 may be obtained from a source other than an associated sequencing platform.

Storage 860 may also store one or more reference genomes 864, and/or system 800 may be in communication with a reference genome database. A reference genome database may be a public database or a private database and may be stored remotely and accessed via the communication interface. The reference genome database may comprise one or more reference genomes.

It will be apparent that various information described as stored in storage 860 may be additionally or alternatively stored in memory 830. In this respect, memory 830 may also be considered to constitute a storage device and storage 860 may be considered a memory. Various other arrangements will be apparent. Further, memory 830 and storage 860 may both be considered to be non-transitory machine-readable media. As used herein, the term non-transitory will be understood to exclude transitory signals but to include all forms of storage, including both volatile and non-volatile memories.

While variant analysis system 800 is shown as including one of each described component, the various components may be duplicated in various embodiments. For example, processor 820 may include multiple microprocessors that are configured to independently execute the methods described herein or are configured to perform steps or subroutines of the methods described herein such that the multiple processors cooperate to achieve the functionality described herein. Further, where one or more components of system 800 is implemented in a cloud computing system, the various hardware components may belong to separate physical systems. For example, processor 820 may include a first processor in a first server and a second processor in a second server. Many other variations and configurations are possible.

According to an embodiment, storage 860 of variant analysis system 800 may store one or more algorithms and/or instructions to carry out one or more functions or steps of the methods described or otherwise envisioned herein. For example, processor 820 may comprise alignment instructions or software 865, merging instructions or software 866, variant calling instructions or software 867, allele-specific expression categorization instructions or software 868, and/or report generation instructions or software 869, among many other algorithms and/or instructions to carry out one or more functions or steps of the methods described or otherwise envisioned herein.

According to an embodiment, alignment instructions or software 865 direct the system to align the DNA and RNA sequence data with a reference genome. The sequence data may be any sequence data from a genetic sample, and may be generated or otherwise obtained by the system. For example, the variant analysis system may comprise a sequencing platform configured to obtain sequencing data from the genetic sample, or may be in communication with or otherwise receive sequencing data generated by another system from the genetic sample. The generated and/or received sequencing data may be stored in a local or remote database for use by the variant analysis system. The generated and/or received sequencing data may comprise a complete or mostly complete genome, or may be a partial genome. For example, the generated and/or received sequencing data may be assemblies, whole genome constructs, incomplete genomes, partial genomes, exomes, and/or any other sequencing data.

The reference genome used by the system for the alignment may be any reference genome, such as a standard reference genome or a reference genome selected from a plurality of possible reference genomes. The reference genome may be stored by the system or may be obtained, retrieved, or otherwise received by the system. According to an embodiment the reference genome is a FASTA file, although many other file types are possible.

Once the system has the sequencing data and a reference genome, the alignment instructions or software 865 direct the system to align the sequencing data with a reference genome. The sequencing data is aligned with the reference genome using any method of alignment, including but not limited to current and future alignment algorithms or methods. There are a variety of different tools available for sequence alignment, including both proprietary and open-source software, and any of these tools may be used to align the plurality of sequencing reads with the reference genome. Accordingly, system 800 may comprise proprietary and/or open-source software or algorithms configured to align the sequencing data with the reference genome. The alignment instructions or software 865 therefore instruct system 800 to generate a genome alignment utilized by other functionality of the system.

According to an embodiment, merging instructions or software 866 direct the system to merge the aligned RNA sequencing data and aligned DNA sequencing data into a single merged alignment file, using any method for merging two or more alignments. According to other embodiments, merging instructions or software 866 direct the system to merge two variant files into a single variant file. For example, at step 132 of the method variants are separately identified in each type of sequencing data, and merging instructions or software 866 direct the system to merge the variants into a single variant compilation. The merging instructions or software 866 may also or alternatively direct the system to validate variants identified in one type of sequencing data using sequencing data of a second type, thereby producing a single merged identification of variants. For example, at step 132 of the method variants are identified in sequencing data of one type (DNA or RNA) and are validated using the sequencing data of the second type (the other of DNA or RNA).

According to an embodiment, variant calling instructions or software 867 direct the system to identify variants in an alignment. Variants may be identified using any variant calling method, including but not limited to Varscan, Samtools, and GATK, among many others. The variant allele calling instructions or software 867 may therefore comprise proprietary and/or open-source software or algorithms. The instructions may direct the system to identify, for example, the location of an allele variant, the variant alleles at that location, and/or the frequencies of the variant alleles at that location. The variant alleles will typically comprise one allele corresponding to the reference genome and a second, different allele.

According to an embodiment, variant allele calling instructions or software 867 direct the system to only identify variants that satisfy a certain threshold, thus being high-confidence variants. The variant calling algorithm may, for example, require that a variant be identified at a minimum frequency such as 25%, 50%, 75%, or any other percentage. This may be dependent upon the read depth of the variant location as described herein. The threshold may be programmed, selected, or otherwise determined by the system and/or by a user. For example, a user may select a frequency threshold via user interface 840, among other input methods.

According to an embodiment, expression characterization instructions or software 868 direct the system to generate or characterize expression for each of at least a plurality of variants within the set of variants, utilizing expression data. According to an embodiment, the generated or characterized expression status comprises one of a plurality of allele-specific expression categorizations comprising expression information for an alternative allele of the variant and expression information for a reference allele of the variant, if there is one. The expression characterization instructions or software 868 can direct the system to generate an output comprise an allele-specific expression categorization (“ase_status”) for each variant, with a categorical variable or identifier that indicates the expression status of the variant. The allele-specific expression categorization may comprise a wide variety of different variables, labels, or identifiers. The allele-specific expression categorization may also comprise the expression or read count of the reference allele (“ase_ref”) and the expression or read count of the alternative allele (“ase_alt”), among other possible information. These and other outputs are possible.

According to an embodiment, report generation instructions or software 569 direct the system to generate a user report comprising information about the analysis performed by the system. For example, a report may comprise an allele-specific expression categorization for each variant, as well as the expression or read count of the reference allele and/or the expression or read count of the alternative allele, among other information. The report may be generated for any format or output method, such as a file format, a visual display, or any other format. A report may comprise a text-based file or other format comprising the reported information.

The report generation instructions or software 869 may direct the system to store the generated report or information in temporary and/or long-term memory or other storage. This may be local storage within system 800 or associated with system 800, or may be remote storage which received the report or information from or via system 800. Additionally and/or alternatively, the report or information may be communicated or otherwise transmitted to another system, recipient, process, device, and/or other local or remote location.

The report generation instructions or software 869 may direct the system to provide the generated report to a user or other system. For example, the variant analysis system may visually display information about one or more of the variants on the user interface, which may be a screen or other display. A clinician or researcher may only be interested in one or several variants, and thus the variant analysis system may be instructed or otherwise designed or programmed to only display information obtained for the one or several variants.

According to an embodiment, the variant analysis system and approach described or otherwise envisioned herein enables a researcher, clinician, or other user to more accurately determine the genotype of the genetic sample, and thus to implement that information in research, diagnosis, treatment, and/or other decisions. This significantly improves the research, diagnosis, and/or treatment decisions of the researcher, clinician, or other user.

While the embodiments described or otherwise envisioned here depict the more common types of -omic data, in particular DNA-Seq and RNA-Seq, the invention is not restricted to the analysis of the covered -omic types. The methods and systems aggregate evidence across different data modalities by taking into account the underlying molecular biology and extensive prior knowledge on their inter-relationships and disease associations. Rather than the means of data generation, it is the biological meaning of the -omic data and the extracted information that matter. Any new types of -omic data not mentioned in this disclosure can either be used to replace the mentioned ones, should they share similar/same biological meaning, or be integrated into our solution framework through the process of information extraction, functional evaluation, and filtering and ranking of variants using the additional layers of functional evidence established on the new data.

Notably, the methods and systems described herein comprise different limitations each comprising and analyzing millions of pieces of information. For example, next-generation DNA sequencing data comprises reads that number in the 100s of millions or even billions. Similarly, according to Illumina, “most [RNA-Seq studies) require 5-200 million reads per sample, depending on organism complexity and size.” Thus, merging the RNA sequencing data and DNA sequencing data into a single merged alignment will comprise millions or even billions of reads each with varying amounts of sequenced nucleotides. Indeed, the entire genome will be covered numerous times depending upon the depth of the RNA and DNA sequencing. This is something the human mind is not equipped to perform, even with pen and pencil. Additionally, once the RNA sequencing data and DNA sequencing data are merged into a single alignment, the system must analyze those millions or even billions of aligned reads to identify variants. This similarly requires billions of points of comparison. Then, the system characterizes RNA-editing and expression status for each of the identified variants using expression data, again requiring millions of points of analysis. These steps comprise millions or billions of points of comparison, something the human mind is not equipped to perform, even with pen and pencil.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/of” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively. While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for characterizing variant RNA editing and/or expression status for a plurality of variants identified from a genomic sample, using a variant analysis system, comprising:
obtaining sequencing data for the genomic sample, the DNA sequencing data comprising a plurality of different variant types and aligned to a reference genome to generate aligned DNA sequencing data;
obtaining RNA sequencing data for the genomic sample, the RNA sequencing data comprising a plurality of different variant types and aligned to the reference genome to generate aligned RNA sequencing data, and wherein the obtained RNA sequencing data further comprises expression data for each variant;
merging the aligned RNA sequencing data and aligned DNA sequencing data into a single merged alignment, wherein each read comprises a source identifier;
identifying, in the single merged alignment, a plurality of variants relative to the reference genome, the plurality of variants comprising a plurality of different variant types, to generate a set of variants;
characterizing, using the expression data, an RNA editing and/or expression status for each of at least a plurality of variants within the set of variants, wherein the expression status comprises one of a plurality of allele-specific expression categorizations comprising expression information for an alternative allele of the variant and expression information for a reference allele of the variant if there is one; and
generating a report comprising the characterized RNA editing and/or expression status for the plurality of variants within the set of variants.

2. The method of claim 1, wherein the plurality of variants are identified using an RNA sequencing data variant calling protocol.

3. The method of claim 1, wherein the plurality of different variant types comprises at least single nucleotide variants, insertions, deletions, copy number variants, and gene fusions.

4. The method of claim 1, wherein the obtained RNA sequencing data comprises gene expression data, transcript expression data, exon expression data, splicing data, and/or allele-specific expression data.

5. The method of claim 1, wherein each of the plurality of allele-specific expression categorizations comprise an identifier describing the expression information for the alternative allele of the variant relative to the expression information for the reference allele of the variant, and wherein there are a plurality of different identifiers.

6. The method of claim 5, wherein the plurality of different identifiers comprise one or more of unexpressed site, unexpressed variant, expressed variant homozygous, expressed variant up regulated, expressed variant down regulated, expressed variant neutral, expressed variant with inconsistency, unexpressed variant with inconsistency, high-confidence RNA editing, and low-confidence RNA-editing.

7. A system for characterizing variant RNA editing and/or expression status for a plurality of variants identified from a genomic sample, comprising:
a reference genome;
DNA sequencing data for the genomic sample, the DNA sequencing data comprising a plurality of different variant types and aligned to a reference genome to generate aligned DNA sequencing data;
RNA sequencing data for the genomic sample, the RNA sequencing data comprising a plurality of different variant types and aligned to the reference genome to generate aligned RNA sequencing data, and wherein the obtained RNA sequencing data further comprises expression data for each variant;
a processor configured to: (i) merge the aligned RNA sequencing data and aligned DNA sequencing data into a single merged alignment; (ii) identify, in the single merged alignment, a plurality of variants relative to the reference genome, the plurality of variants comprising a plurality of different variant types, to generate a set of variants; (iii) characterize, using the expression data, an RNA editing and/or expression status for each of at least a plurality of variants within the set of variants, wherein the expression status comprises one of a plurality of allele-specific expression categorizations comprising expression information for an alternative allele of the variant and expression information for a reference allele of the variant if there is one; and (iv) generate a report comprising the characterized expression status for the plurality of variants within the set of variants; and
a user interface configured to provide the generated report.

8. The system of claim 7, wherein each of the plurality of allele-specific expression categorizations comprise an identifier describing the expression information for the alternative allele of the variant relative to the expression information for the reference allele of the variant, and wherein there are a plurality of different identifiers.

9. The system of claim 8, wherein the plurality of different identifiers comprise one or more of unexpressed site, unexpressed variant, expressed variant homozygous, expressed variant up regulated, expressed variant down regulated, expressed variant neutral, expressed variant with inconsistency, unexpressed variant with inconsistency, high-confidence RNA editing, and low-confidence RNA-editing.

10. A method for characterizing variant RNA editing and/or expression status for a plurality of variants identified from a genomic sample, using a variant analysis system, comprising:
obtaining DNA sequencing data for the genomic sample, the DNA sequencing data comprising a plurality of different variant types and aligned to a reference genome to generate aligned DNA sequencing data;
obtaining RNA sequencing data for the genomic sample, the RNA sequencing data comprising a plurality of different variant types and aligned to the reference genome to generate aligned RNA sequencing data, and wherein the obtained RNA sequencing data further comprises expression data for each variant;
identifying, a plurality of variants in the DNA sequencing data and a plurality of variants in the RNA sequencing data, each of the plurality of variants comprising a plurality of different variant types, to generate a set of DNA variants and a set of RNA variants;
merging the set of DNA variants and the set of RNA variants into a single set of variants, or validating the plurality of variants in the DNA sequencing data or the plurality of variants in the RNA sequencing data with the variants in the other sequencing data type, to generate a single set of variants;
characterizing, using the expression data, an RNA editing and/or expression status for each of at least a plurality of variants within the set of variants, wherein the expression status comprises one of a plurality of allele-specific expression categorizations comprising expression information for an alternative allele of the variant and expression information for a reference allele of the variant if there is one; and
generating a report comprising the characterized expression status for the plurality of variants within the set of variants.

11. The method of claim 10, wherein the plurality of variants are identified using an RNA sequencing data variant calling protocol.

12. The method of claim 10, wherein the plurality of different variant types comprises at least single nucleotide variants, insertions, deletions, copy number variants, and gene fusions.

13. The method of claim 10, wherein the obtained RNA sequencing data comprises gene expression data, transcript expression data, exon expression data, splicing data, and/or allele-specific expression data.

14. The method of claim 10, wherein each of the plurality of allele-specific expression categorizations comprise an identifier describing the expression information for the alternative allele of the variant relative to the expression information for the reference allele of the variant, and wherein there are a plurality of different identifiers.

15. The method of claim 14, wherein the plurality of different identifiers comprise one or more of unexpressed site, unexpressed variant, expressed variant homozygous, expressed variant up regulated, expressed variant down regulated, expressed variant neutral, expressed variant with inconsistency, unexpressed variant with inconsistency, high-confidence RNA editing, and low-confidence RNA-editing.
